(19) United States
(12) Patent Application Publication
(10) Pub. No.: US 2020/0163737 A1
(43) Pub. Date: May 28, 2020

(54) ILLUMINATION ASSEMBLY FOR USE DURING EYE SURGERY

(71) Applicants: THE CLEVELAND CLINIC FOUNDATION, Cleveland, OH (US); LUMITEX, INC., Strongsville, OH (US)

(72) Inventors: Rishi P. Singh, Shaker Heights, OH (US); Joe Dombrowski, Medina, OH (US); Peter Broer, Bratenahl, OH (US); Randolph J. Hagler, Brecksville, OH (US); David G. Felty, Parma, OH (US); Andrew W. Browne, Irvine, CA (US)

(21) Appl. No.: 16/625,163

(22) PCT Filed: Jun. 20, 2018
(86) PCT No.: PCT/US18/38632

§ 371 Date: Dec. 20, 2019

Related U.S. Application Data
(60) Provisional application No. 62/522,386, filed on Jun. 20, 2017.

(51) Int. Cl. A61B 90/35; A61F 9/007

(52) U.S. Cl. CPC A61B 90/35; A61B 3/0008; A61F 9/007

(57) ABSTRACT

An ocular lighting assembly (10) including an anchor (16) and a plurality of light guides (14). The anchor is configured to mount to a surface of an eye (30) and to maintain the position of the plurality of discrete light guides. The plurality of light guides are arranged to guide light from a light source into the interior of the eye.

### RELATED APPLICATIONS

This application claims the benefit of U.S. Application No. 62/522,386 filed on Jun. 20, 2017, that is herein incorporated by reference in its entirety.

### TECHNICAL FIELD

The present disclosure relates generally to a lighting assembly, and in particular to a lighting assembly configured to illuminate an interior of an eye during surgery.

### BACKGROUND

A common challenge with various types of ocular surgeries is illuminating the inside of the eye. This is especially true during vitrectomies where the angle and intensity of the light is important for ensuring that the vitreous is visible to the surgeon. Currently, the primary method for illumination during ocular surgery requires an invasive approach using some version of a light probe. The light probe is inserted into the eye via a port made in the patient's eye and the physician manipulates the probe with one hand, eliminating the potential for bi-manual surgery and adding undue stress on the patient's eye.

### SUMMARY OF INVENTION

A non-invasive lighting assembly is needed that places the light source directly on the surface of the eye without the need for an incision and without the introduction of heat or electricity to the surface of eye. The lighting assembly should also be able to change the angle and intensity of the light to improve the surgeon's ability to distinguish between different tissue types.

The present invention provides an ocular lighting assembly including an anchor for positioning on a surface of an eye and for maintaining the position of a plurality of discrete light guides arranged to guide light from a light source into the interior of the eye.

In one embodiment, the present invention provides a lighting assembly that utilizes light transmitting member technology to distance the light engine from the light delivery area. The light transmitting member is positioned adjacent a surface of the user's eye and is illuminated by a plurality of light sources included in the light engine. By altering the light sources that are turned on, the directionality of the light is controlled. The light transmitting member transmits the proper amount of light intensity at the required angle without introducing heat or electricity to the eye's surface. In particular, the eye may be illuminated by passing light through the sclera and the choroid into the interior of the eye where it is partially absorbed and scattered.

In another embodiment, the present invention provides one or more light sources positioned on an anchor located on a patient's eye. The anchor is positioned between the light sources and the eye and shields the eye from heat generated by the light sources.

The present invention provides for illumination of the internal structure of the eye without the need for an incision and without introducing heat or electricity to the eye. The present invention also enables the angle, intensity, and wavelength of light to be controlled.

According to one aspect of the invention, there is provided an ocular lighting assembly for illuminating an interior of an eye. The ocular lighting assembly includes an anchor and a plurality of discrete light guides. The anchor has a mounting surface corresponding to a contour of an anterior surface of the eye surrounding the pupil of the eye for adjoining the anchor to the eye when the mounting surface is brought into juxtaposition with the anterior surface of the eye. The plurality of discrete light guides supported by the anchor and arranged to guide light from a light source into the interior of the eye when the anchor is adjoined to the eye.

Alternatively or additionally, the plurality of light guides includes an optical fiber and the anchor has a receptacle for receiving and supporting an emitting surface of the optical fiber.

Alternatively or additionally, the emitting surface of the optical fiber extends through the receptacle to the mounting surface of the anchor whereby a light emitting surface of the optical fiber will be in juxtaposition with the eye.

Alternatively or additionally, the anchor is provided with a lens at the end of the optical fiber for focusing light exiting the light guide into the interior of the eye.

Alternatively or additionally, the anchor is in the form of an annulus surrounding an aperture through which a surgeon can view the interior of the eye when the anchor is adjoined to the eye in surrounding relationship with respect to the pupil of the eye.

Alternatively or additionally, the anchor includes openings for allowing passage of instruments through the anchor and into the eye.

Alternatively or additionally, the openings are circumferentially spaced about the anchor.

Alternatively or additionally, the ocular lighting assembly further includes at least one light source for supplying light to the at least one light guide.

Alternatively or additionally, the at least one light source is spaced apart from the anchor and the at least one light guide includes an optical fiber extending from the at least one light source to the anchor.

Alternatively or additionally, the at least one light source is mounted on the anchor.

Alternatively or additionally, the ocular lighting assembly further includes supports configured for: attachment to an external structure for maintaining a position of the anchor relative to the eye and/or supporting the plurality of discreet light guides.

Alternatively or additionally, the supports extend radially from the anchor.

According to another aspect, there is provided an ocular lighting assembly for illuminating an interior of an eye. The ocular lighting assembly includes at least one light source, at least one light guide, and an anchor. The at least one light source configured to emit light. The at least one light guide has a receiving surface configured to receive light emitted by the at least one light source and an emitting surface configured to emit the light received from the at least one light source. The anchor is configured to maintain a position of the emitting surface of the light guide relative to the eye. The anchor has a mounting surface corresponding to a contour of an anterior surface of the eye surrounding the pupil of the eye for adjoining the anchor to the eye when the mounting surface is brought into juxtaposition with the anterior surface of the eye.

Alternatively or additionally, the at least one light source comprises a plurality of light sources. The at least one light guide includes a plurality of said light guides for receiving light from respective light sources. The anchor maintains the emitting surfaces of the plurality of light guides at different positions for illuminating respective sections of the interior of the eye.

Alternatively or additionally, the plurality of light guides are segmented into multiple groups and each group of light guides receives light emitted by one or more of the plurality of light sources.

Alternatively or additionally, each of the groups of light guides receives light from one of the plurality of light sources and interacts with different portions of the anchor.

Alternatively or additionally, a particular light source of the plurality of light sources emits light into at least two of the groups of light guides.

Alternatively or additionally, the plurality of light sources comprise at least two types of light sources and each type of light source emits a different wavelength of light than the other at least two types of light sources.

Alternatively or additionally, the anchor comprises an inner ring and an outer ring and both the inner ring and the outer are fixed with an adhesive to the light guide, such that the light guide is sandwiched between the inner ring and the outer ring

Alternatively or additionally, when the anchor is positioned adjacent the surface of the eye, the anchor is configured to be located between the light guide and the eye.

Alternatively or additionally, when the anchor is positioned adjacent the surface of the eye, the light emitting surface of the optical fiber extends through the receptacle to the mounting surface of the anchor whereby a light emitting surface of the optical fiber will be in juxtaposition with the eye.

Alternatively or additionally, the anchor includes microlens structures configured to control a propagation direction of light rays exiting the anchor.

Alternatively or additionally, the anchor includes a receptacle configured to receive the light emitting surface of the light guide.

The foregoing and other features of the invention are hereinafter fully described and particularly pointed out in the claims, the following description and annexed drawings setting forth in detail certain illustrative embodiments of the invention, these embodiments being indicative, however, of but a few of the various ways in which the principles of the invention may be employed.

Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

FIG. 1 is a schematic diagram of an ocular lighting assembly of the present invention.

FIG. 2 is a perspective view of an exemplary embodiment of the anchor of the ocular lighting assembly.

FIG. 3 is a perspective view, partly broken away in cross section, of the anchor of FIG. 2 placed on an eye.

FIG. 4 is a perspective view of the anchor of FIG. 2 connected to a light transmitting member.

FIG. 5 is a perspective view of an alternative embodiment of the ocular lighting assembly of the present invention.

FIG. 6 is a cross sectional view of FIG. 4 showing interaction between the anchor and the light transmitting member.

FIG. 7 is a cross sectional view of an alternative embodiment of FIG. 6 in which the light transmitting member is positioned adjacent the eye surface.

FIG. 8 is a crosssectional view of anchors having support structures with different angles for receiving the light transmitting members.

FIG. 9 is a perspective view, partly broken away in cross-section, of the two anchors of FIG. 8.

FIG. 10 is a top view of another embodiment of an anchor including supports.

FIG. 11 is a top view of the anchor of FIG. 10 including light guides and a surgical instrument.

FIG. 12 is an perspective view of the anchor of FIG. 10.

FIG. 13 is a perspective view of the anchor of FIG. 12 including light guides and showing an instrument.

FIG. 14 is a close up view of an ocular lighting assembly placed onto an eye.

FIG. 15 is a cross-sectional view of an anchor placed onto an eye and including light guides and showing an instrument.

FIG. 16 is a perspective view, partly broken away in cross-section, of the anchor of FIG. 15 placed on an eye.

### DETAILED DESCRIPTION OF THE INVENTION

The present invention is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

The present invention provides an ocular lighting assembly including an anchor and a plurality of discrete light guides. The anchor (1) has a mounting surface corresponding to a contour of an anterior surface of an eye and (2) supports the light guides.

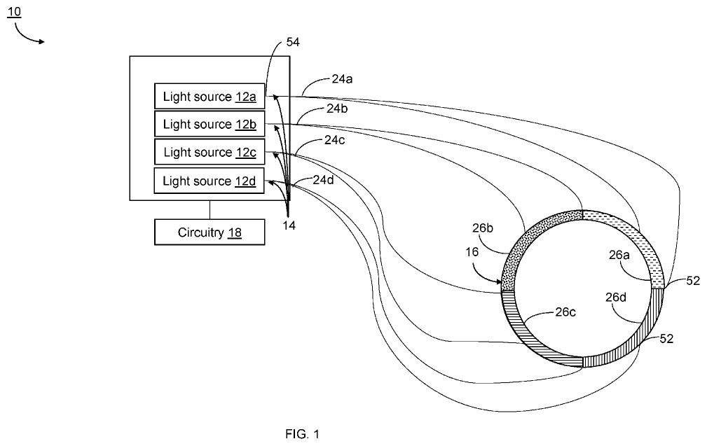

Turning to FIG. 1, an exemplary ocular lighting assembly 10 for illuminating an interior of an eye 30 is shown. The ocular lighting assembly 10 includes a plurality of discrete light guides 14 (also referred to as light transmitting members) and an anchor 16 (also referred to as an anchoring structure) is shown. The anchor 16 has a mounting surface 22 corresponding to a contour of an anterior surface 31 of the eye surrounding the pupil 80 of the eye for adjoining the anchor 16 to the eye 30 when the mounting surface 22 is brought into juxtaposition with the anterior surface 31 of the eye. The light guides 14 are supported by the anchor 16 and are arranged to guide light from a light source 12 into the interior of the eye 30 when the anchor 16 is adjoined to the eye 30.

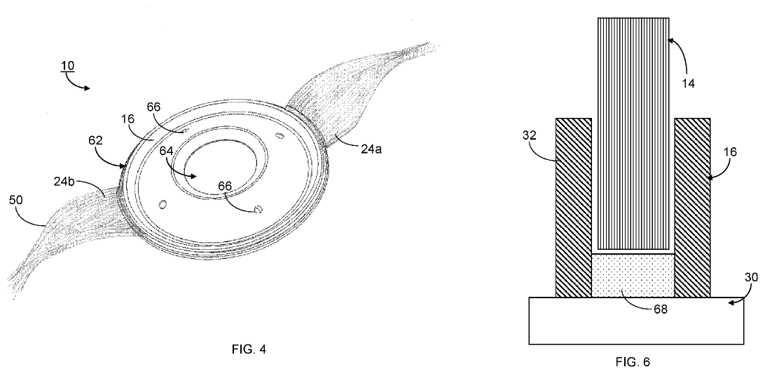

The ocular lighting assembly 10 may also include circuitry 18 and/or at least one light source 12 (e.g., as part of a light engine) for supplying light to the at least one light guide 14. As shown in FIGS. 1 and 4, the light source(s) 12 may be spaced apart from the anchor 16 and the light guide(s) 14 may include an optical fiber 50 extending from the at least one light source to the anchor. Alternatively, the light source(s) 12 may be mounted on the anchor as shown in FIG. 5.

The light guides 14 are configured to propagate light via total internal reflection. Each light guide includes an emitting surface 52 configured to emit the light received from the light source(s) 12 and a receiving surface 54 configured to receive light emitted by the light source(s) 12. That is, the receiving surface 54 may be optically coupled to one or more of the light sources 12, such that the light emitted by the light source(s) 22 is received at the receiving surface 54 of the light guides 14. The light received by the receiving surface 54 of the light guides 14 is propagated via total internal reflection from the receiving surface 54 towards the light emitting end 52 of the light guides 14.

As will be understood by one of ordinary skill in the art, the light guides 14 may include a core surrounded by and in contact with a cladding. The cladding may include one or more layers of materials of lower refractive index than the core. The cladding causes light to be confined to the core of the fiber by total internal reflection at the boundary between the cladding and the core. The light emitting surface 52 may be located at an end of the light guides 14 opposite the receiving surface 54 of the light guides 14. For example, the light emitting surface 52 may also include lensing features configured to alter the trajectory of light leaving the light guides 14.

As will be understood by one of ordinary skill in the art, the light guides 14 may comprise at least one of a flexible fiber optic panel, a molded light guide, a clad flat fiber light guide, parallel optical fibers, braided optical fibers, woven optical fibers, a combination thereof, or any other suitable structure for propagating light. As described above, the propagation of light by total internal reflection may be facilitated by the light guides 14 being formed of a core and cladding surrounding at least a portion of the core, the core having a different index of refraction than the primary member.

As shown in FIG. 3, the light guides 14 may include an optical fiber 50 and the anchor 16 may have a receptacle 32 (also referred to as a support structure) for receiving and supporting an emitting surface 54 of the optical fiber.

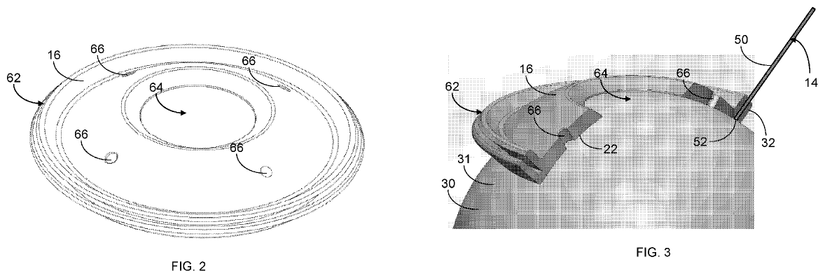

Turning to FIGS. 2 and 3, an exemplary embodiment of the anchor 16 is shown. The anchor 16 is configured to maintain a position of the light emitting surface 54 of the light guide relative to the eye 30. The anchor 16 may be flexible such that the anchor 16 conforms to the surface of the eye. For example, the anchor 16 may be made of a low durometer material.

As will be understood by one of ordinary skill in the art, the anchor 16 may be made of any suitable material capable of supporting the light guides 14 and that does not cause irritation of the eye 30. For example, the anchor 16 may be made from glass, plastic (such as at least one acrylic, polycarbonate, or polystyrene), and/or a ceramic material.

As shown, the anchor 16 may be in the form of an annulus 62 surrounding an aperture 64 through which a surgeon can view the interior of the eye 30 when the anchor 16 is adjoined to the eye 30 in surrounding relationship with respect to the pupil 80 of the eye. The anchor 16 may additionally include one or more openings 66 (e.g., apertures). As described in further detail below, a surgeon may insert instruments 70 through the openings 66 to access the eye 30 during surgery.

The anchor 16 may also include a support structure 32 configured to receive the light emitting surface 52 of the light guides 14. For example, the support structure 32 may comprise a groove configured to receive the light emitting surface 52 of the light guides 14. In FIG. 3, a single optical fiber 50 of the light guide 14 is shown within the support structure 32. In another example, in FIG. 4, two groups 24a, 24b of the light guides 14 are shown connected to the anchor 16.

The light guides 14 may be anchored to the support structure 32 mechanically and/or using an adhesive. For example, the adhesive may be optically transparent to the wavelengths of light emitted by the light engine 12.

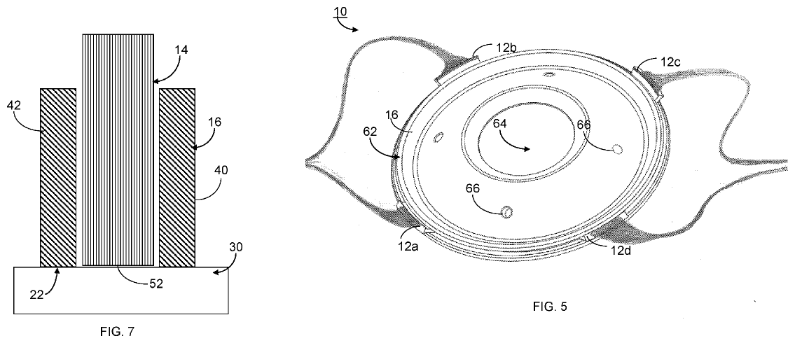

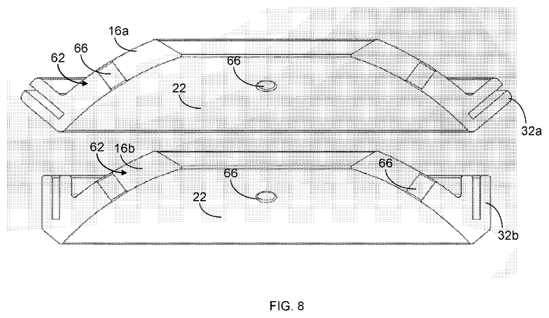

In the embodiment shown in FIG. 6, the anchor 16 is located between the light guides 14 and the eye 30 when the anchor 16 is positioned adjacent the surface of the eye 31. In the embodiment shown in FIG. 7, the light guide 14 is configured to be positioned adjacent the eye 30, such that the anchor 16 is not positioned between the light guide 14 and the eye 30 when the anchor 16 is positioned adjacent the surface of the eye 31. In this embodiment, the light emitting surface 52 of the optical fiber may extend through the receptacle 32 to the mounting surface 22 of the anchor 16 whereby the light emitting surface 52 of the optical fiber 50 will be in juxtaposition with the eye 30.

With continued reference to FIG. 7, the anchor 16 may comprise an inner ring 40 and an outer ring 42. Both the inner ring 40 and the outer 42 may be fixed with an adhesive to the light guides 14, such that the light guide 14 is sandwiched between the inner ring 40 and the outer ring 42. The light guides 14 may be positioned relative to the rings 40, 42, such that the light guides 14 are mechanically sandwiched between the inner ring 40 and the outer ring 42.

The anchor 16 may be configured also to act as an eyelid retractor. That is, the anchor 16 may prevent the patient's eye lids from closing. For example, when placed onto an eye 30, the thickness of the anchor 16 may prevent an eyelid from closing and covering the eye 30.

Turning to FIG. 6, the anchor 16 may be provided with a lens 68 at the light emitting end 52 of the optical fiber 50 for focusing light exiting the light guide 14 into the interior of the eye 30. For example, the anchor 16 may include a microlens structures 68 configured to control a propagation direction of light rays exiting the anchor 16. The microlens structure 68 may comprise a lensing pattern controlling the direction of light as it exits the anchor 16.

Turning to FIG. 4, the ocular lighting assembly 12 may include a plurality of light sources 12 and a plurality of light guides 14 for receiving light from respective light sources. The anchor 16 may maintain the emitting surfaces of the light guides 14 at different positions for illuminating respective sections of the interior of the eye 30. For example, in FIG. 4, approximately half of the light guides 14 are maintained on one side of the anchor 16, with the remainder of the light guides 14 positioned on the opposite side of the anchor 16.

As shown in FIGS. 1 and 4, the light guides may be segmented into groups 24. Each group 24 of light guides 14 may receive light emitted by one or more of the light sources 12. For example, each of the groups 24 of light guides may receive light from one of the light sources 12, with each of the groups 24 of light guides interacting with different portions 26 of the anchor 16. Alternatively, a particular light source 12 of the plurality of light sources may emit light into at least two of the groups of light guides 24.

For example, FIG. 1 shows four groups 24a-24d, where each group 24 receives light from a single light source 12. The different groups 24 may interact with different portions 26 of the anchor 16. Alternatively, a single light source 12 can distribute light to multiple different groups 24. For example, FIG. 1 shows the four groups 24a-24d of the light guides 14 interacting with four different regions 26a-26d of the anchor 16. In this example, the first light source 12a provides light to the upper-right group 24a, the second light source 12b provides light to the upper-left group 24b, the third light source 12c provides light to the lower-left group 24c, and the fourth light source 12d provides light to the lower-right group 24d of the anchor 16. As is described in further detail below, by controlling which light sources 12 are turned on, the directionality of light emitted by the light guides 14 may be controlled.

Each light source 12 may be controllable to emit light within a range of wavelengths (e.g., the visible light range). For example, each of light source 12 may emit light having a wavelength in one of at least two wavelength ranges. As an example, the light sources 12 may include at least two types of light sources 12 and each type of light source may emit a different wavelength of light than the other at least two types of light sources 12.

The circuitry 18 may control operation of the light source(s) 12. For example, each light source 12 may be controllable to emit a particular color of light or a sub-range of a particular color of light.

Each of the light sources 12 may comprise at least one of light emitting diodes (LEDs), organic LEDs (OLEDs), laser sources, incandescent sources, fluorescent sources, or any other suitable source of light.

Light (as used herein) may refer to visible and non-visible light.

The circuitry 18 is configured to control operation of the light source 12. For example, the circuitry 18 may control the light sources 12 that emit light, the wavelength of light emitted by each light source 12, and the intensity of light emitted by each light source 12. The circuitry 18 may receive input from a user input device controlled by a physician. By using the user input device (e.g., a dial, joystick, keypad, etc.), the physician may control the light sources 12 that emit light and, in this way, control the groups 24 of the light guides 14 that receive light and the directionality of light. Similarly, the user input device may be used to control the intensity of light and the wavelength of light emitted by the light engine 12.

As described above, the plurality of light sources 12 may be fixed to the anchor 16 as shown in FIG. 5. For example, the anchor 16 may include multiple light emitting diodes located on the anchor 32. The light source 12 may be connected to a power supply by external wires (as shown). The anchor 16 provides a protective barrier between the eye 30 and the light source 12. For example, the anchor may be configured to protect the eye from heat generated by the light sources 12 and the electricity provided to the light sources 12. That is, the anchor may be made from a material having thermal properties such that they eye 30 is protected from the heat generated by the light sources 12. For example, the anchor may be made from plastic (such as at least one acrylic, polycarbonate, or polystyrene) and/or a ceramic material.

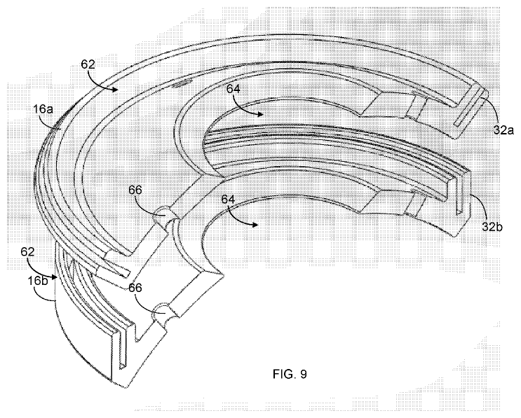

Turning to FIGS. 8 and 9, a cross sectional view of two anchors 16 are shown. As shown, the properties of the receptacle 32 may differ between anchors 16a (or between portions 26 of the same anchor 16), such that an axis of the light guide 14 entering the receptacle 32 points towards different locations in the eye 30. For example, the receptacle 32a is positioned such that an axis of the light guide 14 entering the receptacle 32a points towards a center of the aperture 64. Conversely, the receptacle 32b is positioned such that an axis of the light guide 14 entering the receptacle 32b points towards a peripheral edge of the anchor 16b.

The ocular lighting assembly 10 may be kept in place relative to the eye 30 by attaching the anchor 16 to an eye lid retractor or any other similar instrument. For example, the ocular lighting assembly 10 may be attached to rails above a patient's head. That is, the light guides 14 and/or wires extending from the anchor 16 may be supported by a railing system around the patient.

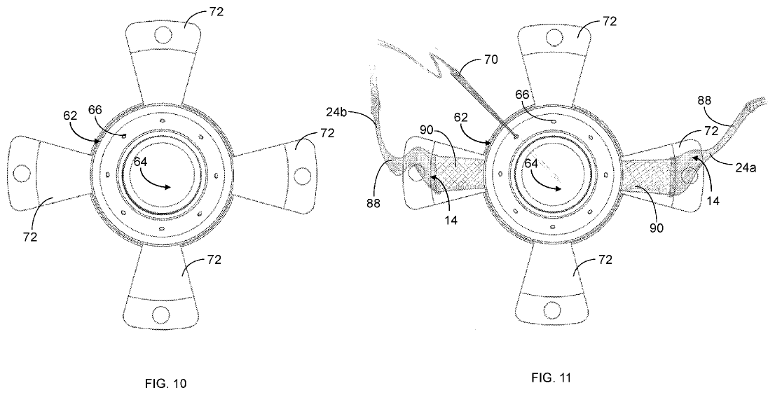

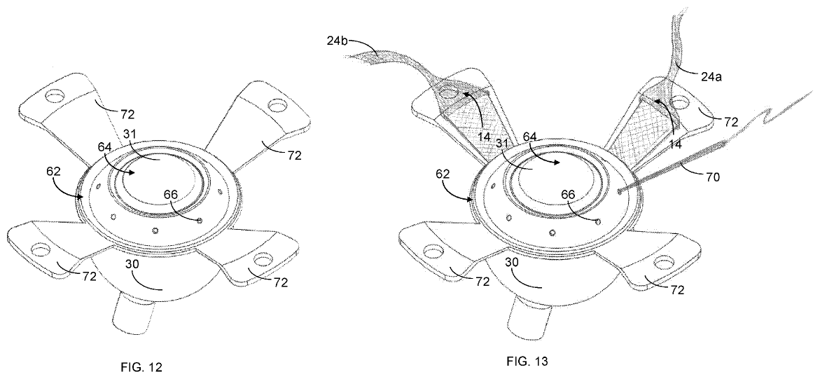

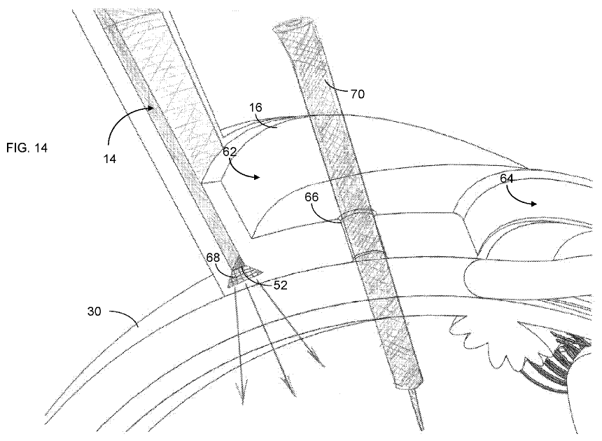

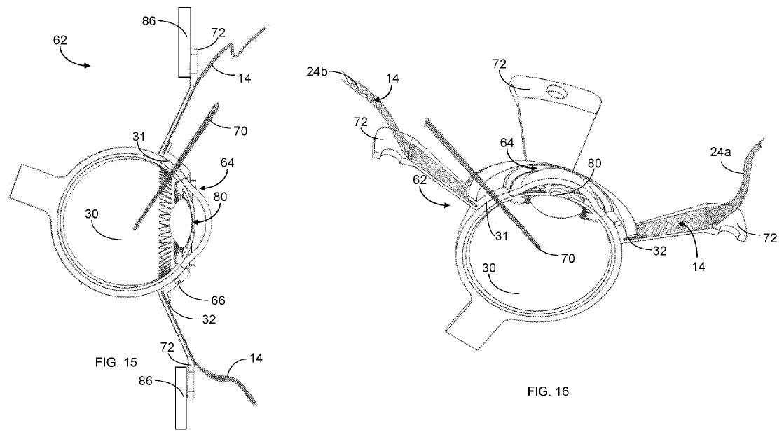

Turning to FIGS. 10-16, the ocular lighting assembly 10 may additionally include supports 72 configured for attachment to an external structure 86 for maintaining a position of the anchor 16 relative to the eye 30. For example, the holes shown in the supports 72 may be used to mount the ocular lighting assembly 10 to the external support. The supports 72 may also be configured for supporting the light guides 14. As shown, the supports 72 may extend radially from the anchor 16.

The supports 72 may be integrally formed with the anchor 16. Alternatively, the supports 16 may be attached to the anchor 16. The supports 72 may be made from the same material or from a different material than the anchor 16. For example, the anchor 16 may be made from plastic and the supports 72 may be made from metal.

As described above and as shown in the figures, the supports 72 may be used to support the light guides 14. For example, the light guide 14 may lie along the supports 72 or the light guide 14 may be fastened to the supports 72 using mechanical fasteners, adhesives, or any suitable method for attaching the light guide 14 to the supports 72.

The portion 90 of the light guide 14 supported by the supports 72 may also differ from other portions 88 of the light guide 14. For example, the portion 88 of the light guide extending between the light source 12 and the anchor 16 may comprise optical fibers in parallel, while the portion 90 of the light guide supported by the support 72 may comprise woven optical fibers. As will be understood by one of ordinary skill in the art, the difference between the portions 88, 90 of the light guide are not limited to parallel and woven optical fibers.

With continued reference to FIGS. 10-16, the anchor may include openings 66 for allowing passage of instruments 70 through the anchor 16 and into the eye 30. For example, the openings 66 may be circumferentially spaced about the anchor 16. While shown in the figures as having a fixed size, the size of the openings 66 may vary (e.g., to permit the entry of instruments 70 of different sizes). The placement and number of openings 66 may also vary. For example, openings 66 may not be equally spaced about the anchor 16, but may instead be concentrated in a particular section of the anchor 16.

As shown, one or more instruments 70, in particular surgical instruments, may be inserted through the openings 66 located in the anchor 16. These instruments 70 may be used to operate on different areas of the eye 30. The instruments 70 may be viewed by a surgeon through the aperture 64.

In one embodiment, the ocular lighting assembly 10 may be integrated into an eyelid retractor. For example, the ocular lighting assembly 10 may include light source(s) 12 located separate from the eyelid retractor and a light guides 14 (receiving light from the light source(s) 12) may engage with the eyelid retractor such that the light guides 14 is repositionable to change the portion of the eye 30 illuminated by light leaving the light guides 14.

As will be understood by one of ordinary skill in the art, the circuitry 18 may have various implementations. For example, the circuitry 18 may include any suitable device, such as a processor, digital signal processor (DSP), Application Specific Instruction Set Processor (ASIP), Application Specific Integrated Circuit (ASIC), Field Programmable Gate Array (FPGA), programmable circuit, integrated circuit, memory and I/O circuits, an application specific integrated circuit, microcontroller, complex programmable logic device, other programmable circuits, or the like. The circuitry 18 may also include a non-transitory computer readable medium, such as random-access memory (RAM), a read-only memory (ROM), an erasable programmable read-only memory (EPROM or Flash memory), or any other suitable medium. Instructions for performing the method described below may be stored in the non-transitory computer readable medium and executed by the circuitry.

Although the invention has been shown and described with respect to certain embodiments, it is obvious that equivalent alterations and modifications will occur to others skilled in the art upon the reading and understanding of the specification. In particular, regarding the various functions performed by the above-described components, the terms used to describe such components are intended to correspond, unless otherwise indicated, to any component which performs the specified function of the desired component (e.g., that is functionally equivalent), even though not structurally equivalent to the disclosed component which performs the function of the herein disclosed exemplary embodiment of the invention. In addition, while a particular feature of the invention may have been disclosed with respect to only one embodiment, such feature may be combined with one or more other features as may be desired and advantageous for any given or particular application.

1. An ocular lighting assembly for illuminating an interior of an eye, the ocular lighting assembly comprising:
an anchor having a mounting surface corresponding to a contour of an anterior surface of the eye surrounding the pupil of the eye for adjoining the anchor to the eye when the mounting surface is brought into juxtaposition with the anterior surface of the eye; and
a plurality of discrete light guides supported by the anchor and arranged to guide light from a light source into the interior of the eye when the anchor is adjoined to the eye.

2. The ocular lighting assembly of claim 1, wherein the plurality of light guides includes an optical fiber and the anchor has a receptacle for receiving and supporting an emitting surface of the optical fiber.

3. The ocular lighting assembly of claim 2, wherein:
the emitting surface of the optical fiber extends through the receptacle to the mounting surface of the anchor whereby a light emitting surface of the optical fiber will be in juxtaposition with the eye; and/or
the anchor is provided with a lens at the end of the optical fiber for focusing light exiting the light guide into the interior of the eye.

4. The ocular lighting assembly of any preceding claim, wherein the anchor is in the form of an annulus surrounding an aperture through which a surgeon can view the interior of the eye when the anchor is adjoined to the eye in surrounding relationship with respect to the pupil of the eye.

5. The ocular lighting assembly of any preceding claim, wherein the anchor includes openings for allowing passage of instruments through the anchor and into the eye.

6. The ocular lighting assembly of claim 5, wherein the openings are circumferentially spaced about the anchor.

7. The ocular lighting assembly of any preceding claim, further comprising at least one light source for supplying light to the at least one light guide.

8. The ocular lighting assembly of claim 7, wherein:
the at least one light source is spaced apart from the anchor and the at least one light guide includes an optical fiber extending from the at least one light source to the anchor; or
the at least one light source is mounted on the anchor.

9. The ocular lighting assembly of any preceding claim, further comprising supports configured for:
attachment to an external structure for maintaining a position of the anchor relative to the eye; and/or
supporting the plurality of discreet light guides;
wherein the supports extend radially from the anchor.

10. An ocular lighting assembly for illuminating an interior of an eye, the ocular lighting assembly comprising:
at least one light source configured to emit light;
at least one light guide having a receiving surface configured to receive light emitted by the at least one light source and an emitting surface configured to emit the light received from the at least one light source; and
an anchor configured to maintain a position of the emitting surface of the light guide relative to the eye, the anchor having a mounting surface corresponding to a contour of an anterior surface of the eye surrounding the pupil of the eye for adjoining the anchor to the eye when the mounting surface is brought into juxtaposition with the anterior surface of the eye.

11. The ocular lighting assembly of claim 10, wherein:
the at least one light source comprises a plurality of light sources;
the at least one light guide includes a plurality of said light guides for receiving light from respective light sources; and
the anchor maintains the emitting surfaces of the plurality of light guides at different positions for illuminating respective sections of the interior of the eye.

12. The ocular lighting assembly of claim 11, wherein the plurality of light guides are segmented into multiple groups and each group of light guides receives light emitted by one or more of the plurality of light sources.

13. The ocular lighting assembly of claim 12, wherein:
each of the groups of light guides receives light from one of the plurality of light sources and interacts with different portions of the anchor; or
a particular light source of the plurality of light sources emits light into at least two of the groups of light guides.

14. The ocular lighting assembly of any of claims 10-13, wherein:
the plurality of light sources comprise at least two types of light sources and each type of light source emits a different wavelength of light than the other at least two types of light sources; and/or
the anchor comprises an inner ring and an outer ring and both the inner ring and the outer are fixed with an adhesive to the light guide, such that the light guide is sandwiched between the inner ring and the outer ring

15. The ocular lighting assembly of any of claim 16, wherein:
when the anchor is positioned adjacent the surface of the eye, the anchor is configured to be located between the light guide and the eye; or
when the anchor is positioned adjacent the surface of the eye, the light emitting surface of the optical fiber extends through the receptacle to the mounting surface of the anchor whereby a light emitting surface of the optical fiber will be in juxtaposition with the eye.

16. The ocular lighting assembly of any of claims 12-17, wherein:
the anchor includes microlens structures configured to control a propagation direction of light rays exiting the anchor; and/or
the anchor includes a receptacle configured to receive the light emitting surface of the light guide.
